(19) United States
(12) Patent Application Publication
(10) Pub. No.: US 2008/0257735 A1
(43) Pub. Date: Oct. 23, 2008

(54) Microfluidic Device for Enabling the Controlled Growth of Cells and Methods Relating to Same

(75) Inventors: Noo Li Jeon, Newport Beach, CA (US); Anne M. Taylor, San Marino, CA (US); Carl W. Cotman, Tustin, CA (US); David H. Cribbs, Newport Beach, CA (US)

Correspondence Address: DALINA LAW GROUP, P.C., 7910 IVANHOE AVE. #325, LA JOLLA, CA, 92037, US

(73) Assignee: REGENTS OF THE UNIVERSITY OF CALIFORNIA, Oakland, CA (US)

(21) Appl. No.: 11/575,908

(22) PCT Filed: Sep. 26, 2005
(86) PCT No.: PCT/US2005/034792

§ 371 Date: Jan. 30, 2008

Related U.S. Application Data
(60) Provisional application No. 60/613,061, filed on Sep. 24, 2004.

(51) Int. Cl. C07K 1/26

(52) U.S. Cl. 204/453

(57) ABSTRACT

A multi-compartment microfluidic device for enabling fluidic isolation among interconnected compartments and accomplishing centrifugal positioning and/or patterned substrate positioning of biological specimens. Includes micropatterned substrate coupled with optically transparent housing allowing imaging. Housing includes microfluidic region having entry reservoir for accepting first volume of fluid and additional microfluidic region(s) having a second entry reservoir for accepting second volume of fluid less than first volume of fluid to create hydrostatic pressure. A barrier region that couples the microfluidic region with the second microfluidic region enables biological specimen(s) to extend across the microfluidic, barrier region and second microfluidic region. The barrier region includes embedded microgroove(s) having width and height enabling second volume of fluid to be fluidically isolated from first volume of fluid via hydrostatic pressure maintained via the embedded microgroove(s). Cells are aligned to a chosen location using a centrifuge or patterned substrate techniques.

This application claims priority from U.S. Provisional Patent Application Ser. No. 60/613,061 entitled “A MICROFLUIDIC DEVICE FOR ENABLING THE CONTROLLED GROWTH OF CELLS AND METHODS RELATING TO SAME” filed on Sep. 24, 2004 the specification of which is hereby incorporated herein by reference. This application is a continuation in part of U.S. patent application Ser. No. 10/605,537 entitled “MICROFLUIDIC DEVICE FOR NEUROSCIENCE RESEARCH” filed on Oct. 6, 2003 which takes priority from U.S. Patent Application Ser. No. 60/416,278 entitled “MICROFLUIDIC MULTI-COMPARTMENT DEVICE FOR NEUROSCIENCE RESEARCH” filed Oct. 4, 2002 the specifications of which are both hereby incorporated herein by reference.

### BACKGROUND OF THE INVENTION

1. Field of the Invention

One or more embodiments of the invention relate to the filed of nano-scale devices and more specifically relate to a microfluidics device for enabling the controlled growth of cells and methods relating to the use and manufacturing of such devices.

2. Description of Related Art

Neurons extend processes in order to form connections and transmit information. These processes are called axons and dendrites (together, these processes are called neurites). When a dendrite of one neuron and an axon of another neuron connect, they make a synapse. In many neurodegenerative diseases and in spinal cord injury, axons and synapses are damaged; a cell culture model is useful for investigation into these areas of research. In typical cell culture it is difficult to distinguish axon from dendrite, and fairly impossible to simulate microenvironments encountered along axons, dendrites or synapses. In a Petri dish for example there is no way to prevent cells from combining and hence it is difficult and in some cases impossible for even a scientifically trained person to isolate the cells for purposes of performing tests or exposing the cells to different solutions. Hence there is a need for a microenvironment that allows for the controlled growth and use of neurons and other cellular structures. The inventions described herein solves these and other problems inherent in the prior art through the use of various devices and methods for obtaining control over the growth of various biological structures such as neurons or other cell types.

Existing devices called, Campenot chambers, provide a basic structure for growing neurons. The Campenot chamber makes use of a tissue culture dish that is coated with collagen. Parallel lines, spaced 200 um apart, are scratched along the surface of the dish. A three-compartment Teflon piece is sealed to a Petri dish with silicone grease and neurons are plated in the small central chamber of the Teflon piece. Nuerites grow outwards into the two other compartments on either side, aligning parallel to the scratches. Variations of the Campenot chamber have been used in studies of various types of long projection neurons. However the Campenot chamber and its variations do not work well when used to culture cortical and hippocampal neurons.

Ivins, et al. developed a chamber designed for cortical and hippocampal neuron cultures using a relatively short barrier distance (150 um versus 300 um in the classic Campenot chamber). These chambers use a glass coverslip fixed to hemisected Teflon tubing using Sylgard 184 (Dow Corning, Corning N.Y.). A small amount of silicone vacuum grease is applied to the bottom of the converslip using a dissecting microscope and the whole apparatus is placed on the tissue culture dish. Neurites extend through the vacuum grease barrier between the polystyrene and the coverslip, if the vacuum grease barrier is sufficiently thin. A problem with these devices is that the process of making the chambers is laborious and their successfulness is directly related to the skill level of the individual using the device. Additionally, there is no alignment of neurons and the apparatus is not compatible with live cell imaging, thus, the effects of insults were observed only after the cells were fixed.

It is also desirable to position cell within the microfluidic device as needed. It is also desirable to position cell within the microfluidic device as needed. Several groups have reported successful culture and manipulation of mammalian and insect cells inside microfluidic devices. For example, one techniques makes use of multiple laminar flows to perform patterned cell deposition in capillary networks. Another attractive aspect is the ability to use multiple laminar streams to selectively expose part of the cell to different chemical reagents and investigate the cellular responses. If methods are available to place cells preferentially within microfluidic channels, such partial treatment of cells using multiple laminar flow streams would be more amenable to high-throughput investigations.

Recently, several examples have been reported where hydrodynamic, dielectrophoretic, and electroosmotic and electrophoretic forces have been used to trap, transport and sort cells. It is feasible for instance to use electrical and optical addressing with microelectrodes to trap and place biological samples over large areas

In order to overcome these and other limitations present in the prior art there is a need for an improved device that allows for the controlled positioning and growth of cells and the application of different compounds to different areas of the cell.

### SUMMARY OF THE INVENTION

One or more embodiments of the invention are directed towards a multi-compartment microfluidic device for enabling fluidic isolation among interconnected compartments and accomplishing centrifugal positioning and/or patterned substrate positioning of biological specimens within the device. One or more devices comprise a micropatterned substrate coupled with an optically transparent housing for purpose of imaging the biological specimens grown within the device. The optically transparent housing comprises a first microfluidic region having a first entry reservoir for accepting a first volume of fluid and further comprises at least one additional second microfluidic region having a second entry reservoir for accepting a second volume of fluid that is less than the first volume of fluid to create hydrostatic pressure. In some cases additional microfluidic regions such as a center region are introduced. A barrier region that couples the first microfluidic region with the second microfluidic region to enables a biological specimen to extend across the first microfluidic region, the barrier region, the second microfluidic region, and optionally the center region. The barrier region comprises at least one embedded microgroove having a width and height that enables the second volume of fluid to be fluidically isolated from the first volume of fluid via hydrostatic pressure maintained via the at least one embedded microgroove. Cells are aligned to a chosen location through the use of centrifugal force or through patterned substrate techniques.

One or more embodiments of the invention are directed to a microfluidics-based multi-compartment culture chamber for neurons (e.g., cortical and hippocampal nurons) that polarizes and isolates axons separately from cell bodies and dendrites. This microfluidic culture chamber is the first easily reproducible chamber to culture cortical and hippocampal neurons that does not require trophic factors to guide axonal growth. Since neurons are polarized and axons are isolated to one compartment, questions involving axonal transport, synaptic Development, and axonal degeneration can readily be addressed using this method.

Potential applications of this method to research in neurodegenerative diseases, spinal cord injury and fundamental biological questions are described. In neurodegenerative diseases such as Alzheimer's disease (AD), synaptic degeneration and deficits in axonal transport appear to play an important etiological role. Co-cultures of wild-type cells with neurons from various transgenic models of AD allow isolated study of synaptic growth and degeneration. Axonal responses to candidate proteins implicated in the pathogenesis of AD can also be studied. This method has applications as an in vitro model for demyelinating conditions such as multiple sclerosis by co-culturing oligodendrocytes only within the axonal compartment; this will more faithfully model conditions within white matter tracts in vivo. The microfluidic culture chamber can also be readily applied to the study of spinal cord injury and regeneration by severing axons and examining potential growth promoting or inhibitory compounds. Other cell-types can be applied to the injured axons with or without concurrent application of these compounds to neuronal cell bodies. Other fundamental biological questions regarding synaptogenesis, axonal growth, and both retrograde and anterograde cell signaling and transport can also be examined using this model.

The microfluidic culture chambers are fabricated in an optically transparent polymer, PDMS [poly(dimethylsiloxane)], using microfabrication and soft lithography techniques. The PDMS chamber, placed on a polylysine coated glass coverslip, allows various microscopy techniques to be used, including differential interference contrast (DIC), epifluorescence, confocal and multi-photon microscopy. A barrier with embedded microgrooves separates the somal and the axonal compartments, allowing the compartments to be fluidically isolated but physically connected. When the dissociated primary neurons are plated into the somal compartment, neurons extend processes through the microgrooves in the barrier into the axonal compartment. Since axons tend to grow longer and straighter than dendrites, we adjusted the geometry of the chamber to allow only axons through the barrier. The processes extending from barriers equal to 450 μm or more are axons.

Using the devices described herein primary rat (E18) cortical and hippocampal neurons have been successfully cultured for over 3 weeks in the microfluidic culture chambers. Mouse cultures have also been used successfully. The viability and morphology of the neurons are similar to controls grown on tissue culture dishes. Chambers with barriers greater than 450 μm isolate axons exclusively in the axonal compartment. The isolation of axons can be confirmed by immunostaining with microtubule-associated proteins found in axons (MAP5) and dendrites (MAP2). In addition, when glutamate can be isolated to the axonal compartment, CREB can be not activated in cell bodies, indicating that there are no dendrites in the axonal compartment which could active CREB via exposure to glutamate. This finding provides further evidence that axons are microfluidically isolated within these chamber cultures. Within 3 days in vitro, robust growth of axons into the axonal compartment is observed. We describe 3 models using this method for studying (1) presynaptic differentiation, (2) demyelination, and (3) spinal cord injury and regeneration. Immunostaining with synapsin, synaptophysin, SNAP-25 and Rab 3A show that synaptic-like connections form between presynaptic hippocampal neurons and human SH-SY5Y cells. We show that oligodendrocytes can be co-cultured in the axonal compartment to study mechanisms of myelination and demyelination. Finally, we show that we can use this method to sever CNS axons in order to use the chamber as a model for spinal cord injury.

### BRIEF DESCRIPTION OF THE DRAWINGS

FIG. 1 shows a detailed schematic outline of the procedure for micropatterning cells inside a microfluidic device using cell-adhesive or non-adhesive substrates.

FIG. 2 shows an example of a microfluidic device configured in accordance with one or more embodiments of the invention.

FIG. 2a illustrates a photograph of neurons grow in accordance with one or more embodiments of the invention.

FIG. 3 shows patterned HUVEC, MDA-MB-231 human breast cancer cells and NIH 3T3 mouse fibroblasts cultured for 5-48 h on patterned Petri dishes grown in accordance with one or more embodiments of the invention.

FIG. 4 shows rat cortical neurons grown using the patterned microfluidic device configured in accordance with one or more embodiments of the invention.

FIG. 5 illustrates dissociated cells plated on the PLL treated substrates and in the microfluidic channels configured in accordance with one or more embodiments of the invention.

FIG. 6 illustrates a schematic drawing of microfluidic “module” as used where cell suspensions are introduced into the middle channel and centrifugal, hydrodynamic, and gravitational forces are applied before the cells settled and attached to the substrate; three compartments are separated by barriers (100 μm wide) that have embedded microgrooves (3 μm high and 10 μm wide), that act as a filter for cells but allow fluid transport.

FIG. 7 shows the positioning of a set of devices within a photoresist spinner in accordance with one or more embodiments of the invention.

FIG. 8 illustrates a schematic illustration of positioning cells inside microfluidic channels by centrifugal force where (b) NIH 3T3 mouse fibroblasts are positioned along a wall inside microfluidic channel with ˜20-25 g of RCF; the number of cells positioned along the wall can be a function of cell suspension density indicated in the upper left corner; Fluorescent micrographs show live cells that were stained with fluorescent probe, calcein AM; White dotted lines indicate channel boundaries not visible with fluorescence microscopy.

FIG. 9 shows the application of two or more forces results in more reproducible cell placement along a wall. Suspension of dissociated NIH 3T3 mouse fibroblasts can be introduced into the microfluidic device with and without external forces. When no external force can be applied, cells randomly attached on the substrate inside the microfluidic channel. FIG. 9a shows cells 1 hour after random loading. FIG. 9c shows cell placement when combination of gravitational and hydrodynamic force can be used while loading the cell. FIG. 9e shows the result for combination of hydrodynamic force, gravitational force and aspiration. Inset figures show fluorescence micrographs of viable cells stained with calcein AM, a live cell marker. Micrographs taken after 24 hours are shown in FIGS. 9b, d, and f.

FIG. 10 illustrates primary rat cortical neurons that were successfully positioned and cultured for over 7 days inside the microfluidic devices in accordance with one or more embodiments of the invention. The fluorescence micrographs show calcein AM stained, viable cells that were positioned by (a) combination of gravitational and hydrodynamic forces, (b) combination of hydrodynamic, gravitational force and aspiration, and (c) centrifugal force. (d) Phase-contrast micrograph and fluorescence micrograph (inset) of neurons positioned along a wall with centrifugal force and cultured for 7 days in vitro on micropatterned cell adhesive PLL substrate.

FIG. 11 illustrates gravity assisted cell positioning for chemotaxis assay. Metastatic breast cancer cells, MDA-MB 231, were positioned along a wall to align them before exposing them to EGF (chemoattractant) gradient. (a) Fluorescence micrograph of EGF gradient (indirectly visualized with FITC-dextran) and a plot of the fluorescence intensity profile. (b) Differential interference contrast images of migrating cells at 0 and 3 h. (c) Superimposed migration tracks of 20 randomly selected cells from the flat region (control) and steep EGF gradient region.

### DETAILED DESCRIPTION OF THE INVENTION

Embodiments of the invention are directed to microfluidic devices for enabling the controlled growth of various cell types (e.g., neurons) and methods relating to the use and production of such microfluidic devices. One or mores aspects of the invention relate to microfluidic device(s) that enhance growth, polarize, and isolate axons, dendrites, and/or synapses. These microfluidic devices comprise at least two segments disposed against one another (e.g., via a conformal or covalent bond). In at least one embodiment of the invention one or more segments are made by placing an optically transparent material on a flat substrate. The basic design implemented in one or more embodiments of the invention comprises two or more compartments separated by a physical barrier embedded with multiple microgrooves (e.g., two or more).

Neurons represent an excellent cell type to illustrate the concept of selective isolation and treatment and are therefore used herein for purpose of example. Those of ordinary skill in the art, however, will recognize that neurons are a test case and that the device described herein has applicability to other types of cells or biological type applications. The device can, for instance, also be adopted for use with cancer cells and is hence not limited solely to being used for neurons.

Neurons can be added to one or more of the compartments and after a certain time threshold (e.g., a couple days) neurites grow through the microgrooves connecting two compartments. A benefit to directing the growth of neuritis through these microgrooves is that parts of the neurites can be isolated in a compartment within the device. The small size of the microgrooves provides increased resistance to fluidic flow. One compartment can be isolated from the others by hydrostatic pressure, meaning that a chemical microenvironment can be established and thereby enable the application of different chemical solutions to the various parts of the cell.

Other benefits to the device include the ability to create one or more compartments containing exclusively axons, thereby resulting in polarization. For instance, since axons grow longer and straighter than dendrites, the width of the physical barrier can be lengthened to allow only axons to successfully make it to the adjoining compartment.

In one or more embodiments of the invention dendritic growth is enhanced by shortening the physical barrier, by micropatterning (referred to herein as “speed bumps”), and/or by using dendrite-enhancing substances (e.g., semaphorin). A compartment containing targeted synaptic connections between dendrites and axons can be achieved by combining the features intended to isolate axons and enhance dendritic growth within one device.

The invention does not require the use of growth factors for neuritic (dendritic or axonal) growth; although it is possible to use growth factors if desired. In some cases it may advantageous to view the cells grown within the device and hence the devices are designed to permit visibility to the cells. For instance, the invention can use phase contrast imaging and differential interference contrast imaging with the device.

The microfluidic devices described herein is designed to allow biochemical analyses (e.g., PCR, Western blot) of cell bodies, axons, dendrites, and synapses. This benefit is accomplished in one or more embodiments of the invention through the use of compartments designed in such a way as to optimize the percentage of neurons with processes isolated in an adjacent compartment, allowing transport studies to be performed (e.g., if a chemical insult is isolated to the axons, a change in the cell bodies would be able to be detected using PCR and/or Western blotting). Additionally, the chamber geometry can be adjusted so that there is enough cellular material to allow biochemical analyses. Variations in the geometry of the chamber are contemplated as being with the scope and spirit of the invention and many different variations in design accomplish the same basic results; that being the ability to fluidically isolate parts of the cells and expose the isolated part of the cell to different microenvironments.

Cellular microenvironments can be established by plating various other cell types within a compartment. In the context of neuroscience research, a particularly useful case involves using the devices for myelination/demyelination studies where oligodendrocytes are co-culture in the axonal compartment.

Another aspect of the invention describe herein relates to a method for simulating injuries to the central nervous system and performing quantitative analyses on these injured cells. For example, by severing axons isolated to one compartment of the above-mentioned device using suction spinal cord injury can be simulated. Once the axons are removed various chemical and/or cellular microenvironments can be simulated to observe and analyze regeneration.

It is possible to create patterns of adhesive proteins (e.g., polylysine) on portions of the various devices using plasma based dry etching. In this method, a substrate is coated with an adhesive protein, then a raised patterned mold is placed on the substrate and exposed to plasma. The adhesive protein remains on the substrate only in areas in contact with the mold. Using this patterning technique, one or more embodiments of the invention are able to implement a method for micropatterning a substrate, bond, and sterilizing microfluidic devices in a single step. Cells can be selectively placed within a device using centrifugal, gravitional, hydrodynamic forces, or a combination. This is done before cells attach strongly to the substrate. Cell positioning allows that all cells get exposed to the same level of chemical at the start of the experiment helping standardize cellular response.

Fluid flow through a microfluidic channel is maintained in one embodiment of the invention using a passive pumping method based on evaporation. When one reservoir is smaller than the other reservoir connecting a channel, the ratio of evaporation to volume is increased in the smaller reservoir leading to fluid flow from the large reservoir to the smaller, hence passive pumping within the channel is established. This is useful because it does not require an outside pump. A slow flow of medium often enhances cell growth because used medium and nutrients are replaced with new medium.

It is possible to utilize the microfluidic device to polarize, isolate, and analyze different cell parts in open culture. For instance, the device has applicability in evaluating axons, dendrites and synapses in open culture. In this manner, neurons can be accessed using micropipetting techniques. This chamber make use of fluidically isolated compartments that are joined via microgrooves. However, in at least one embodiment the device comprises a substrate containing a physical barrier with open microgrooves to guide neurite growth. A top piece containing the compartments and a solid physical barrier is aligned onto the substrate. Cells are added to the fully assembled device. Once the cells attach to the substrate, the top piece can be lifted off, allowing access to the neurons. This device is beneficial for looking at calcium imaging and electrophysiology. The device can configured to enable access to the microgrooves or other parts of the chamber as desired by the user.

Each of the devices described herein facilitate the study of chemical and/or cellular microenvironments within the brain, synaptogenesis, synaptic degeneration, transport along neurites, local protein synthesis, myelination/demyelination, and spinal cord injury. The devices can be used as models for myelination/demyelination in the central nervous system as well as a model for spinal cord regeneration. One advantage of the microfluidic devices is that it enables for efficient testing of drugs. For instance, a pharmaceutical company could use the devices described here or variations thereof to test drugs related to spinal regeneration, neurodegenerative diseases that affect axons and synapses, diseases such as cancer that spread through raid cell growth, or other diseases where a cells function and/or behavior impacts the course of the disease.

One or more embodiments of the invention relate to microfluidic devices for enabling the controlled growth of cells and methods relating the use and production of such devices. Various devices and methods are contemplated as falling with the scope of the invention. For instance, embodiments of the invention make it possible to construct devices and methods for enhancing growth, polarizing, isolating, and aiding analysis of neuronal processes, both axonal and dendritic, and for isolating and aiding analysis of associated neurons. Embodiments of the invention are also directed at devices and methods for promoting targeted synaptic connections; devices and methods for creating chemical and/or cellular microenvironments along neurons; device and method for simulating spinal cord injury; devices and methods for patterning cell adhesive proteins within above-mentioned devices. Other embodiments are directed to one or more methods for surface patterning, bonding, and sterilization of above-mentioned devices in one or more steps; methods for placement of cells and devices and methods for passive pumping within the various microfluidic devices.

There are various aspects of the device that are unique. For instance, these devices provide the ability to localize cell bodies to one compartment and the ability to localize processes to one compartment. The design of the microgrooves which allow neurites or other cellular growths to grow through them and enhance their growth. The devices also provide one or more of the following: the ability to isolate chemical microenvironments by using hydrostatic pressure between compartments, the ability to co-culture other cells to simulate cellular microenvironments, the ability to polarize the axons, meaning the direction of axonal transport is established, the ability to do biochemical analysis on the axons, dendrites, and cell bodies, the ability to direct and isolate synapses, the ability to simulate spinal cord injury by severing axons, the ability to have myelinated axons in a compartment, the ability to pattern the adhesive protein substrate using plasma based dry etching, the ability to pattern the adhesive protein substrate, bond and sterilze the device in a single step, and the ability to access neurons within the device (e.g., by micropipette techniques). The devices also enable various techniques for cell placement and passive pumping using evaporation.

The dimensions of the compartments within the device are designed for optimal growth of the neurons. The dimensions of the microgrooves within each microfluidic device allow and guide neuritic growth without allowing dissociated cell bodies through. Reservoirs containing cell culture medium connect each compartment which allow nutrient and gas exchange and minimize evaporative losses. Dissociated neurons are pipetted into the somal compartment and can enter the compartment by capillary action. The width of a physical barrier within the device can be designed to allow only axons or other cell parts (e.g., a cytoplasmic domain of a cancer cell) to enter adjacent compartment. Adjusting the width of the physical barrier, substrate patterning, and dendritic enhancing compounds can be used to enhance dendritic growth into a dendritic compartment. Controlling the various characteristics of the physical barrier allows the creation of devices that can promote targeted synaptic connections within a defined test region. Substrate micropatterning may used inside the devices to guide neuritic growth. The dimensions of the somal compartment can be adjusted such that a high percentage of neurons in the somal compartment have neurites isolated in the adjacent compartment which allows for biochemical analyses on their connecting neurons. Other cell types can be co-cultured in and isolated to any of the compartments. Hydrostatic pressure is used in one or more embodiments of the invention to chemically isolate one compartment for several hours. Neurites can be severed and removed from one compartment. Neurites, dendrites, axons, and cell bodies can be removed for biochemical analyses. Axons can be removed from the isolated axonal compartment without detachment of cell bodies. Chemicals and cells can be isolated to regenerating axons. Plasma is used to dry etch an adhesive protein layer in order to create micropatterns on the substrate surface. Micropatterning, bonding, and sterilization can be combined into one step to assemble microfluidic devices. Centrifugal, gravitational, and/or hydrodynamic forces can be used alone or in combination to place cells in a microfluidic channel. Passive pumping is performed using evaporation. Open culture devices for access to individual neurons. Co-culture of axons and oligodendrocytes for a model of myelination/demyelination. Co-cultures of transgenic and transfected neurons in device. Co-cultures with other cell types in device.

A “master” used to replica mold the devices can be made using photolithography. The “master” has two layers of the negative epoxy photoresist SU8 on a silicon wafer. In at least one instance the device is made from PDMS, glass or tissue culture dish substrates are coated with polylysine, and the PDMS mold is conformally bonded to the glass or tissue culture dish.

There are various innovative features incorporated into the device. For instance, the chamber dimensions are adjusted for optimal growth and culturing of neurons. The physical barrier within the device can be embedded with microgrooves. The width of the physical barrier which can be adjusted for axonal growth or for enhancing dendritic growth. Dendritic enhancing surface patterns or dendrite enhancing compounds can be used to promote dendritic growth into a compartment within the device. The device can promote targeted synaptic connections within a defined test region and isolate chemicals to one compartment for several hours using hydrostatic pressure. The device is also capable of co-culturing other cell types, transgenic cells, or transfected cells in a compartment. The device also provides a mechanism for precisely severing axons, removing cell bodies, axons, and neurites for biochemical analyses, and isolating chemicals and/or cells to regenerating axons. The devices are generated using a novel method of micropatterning using plasma and can be created in one step via a unique method for micropatterning, bonding, and sterilizing microfluidic devices. Centrifugal, gravitational, and/or hydrodynamic forces alone or in combination can be used to place cells in a microfluidic channel. The device also enables passive pumping using evaporation and provides a open culture devices for access to individual neurons. Co-culture of axons and oligodendrocytes for a model of myelination/demyelination. Co-cultures of transgenic and transfected neurons in device. Co-cultures with other cell types in device.

Alternative ways to implement the invention include, but are not limited to, at least the following: a)_The device could be made using another optically transparent material (e.g., PMMA). b) The device could be fabricated using another technique besides replica molding, such as injection molding. c) The glass or plastic substrates could be coated with another extracellular matrix protein, other than polylysine. Instead of tissue culture dish you could use plastic. Instead of presynaptic neurons, you could use “their connecting neurons”. The invention can also use pre-assembled device and substrate and materials such as PMMA. The use of microelectrodes is also feasible. The invention can be used for other neuronal types, such as spinal cord neurons. Invention could also be modified to create neuronal circuits and for use with microelectrode arrays. One key aspect of the invention comprises the dimensions and aspect ratios of the invention. In certain situations (not all situations) these dimensions and aspect ratios are required for the device to function. The device must also be made via a biocompatible material that enables cell growth and viability.

Patterning Inside Microfluidic Devices

One or more embodiments of the invention are directed to plasma-based dry etching method that enables patterned cell culture inside microfluidic devices. The plasma-based dry-etching method enables patterning, fluidic bonding and sterilization steps to be carried out in one or more steps. It is possible, for instance, using the described patterning technique to pattern cell-adhesive and non-adhesive areas on the glass and polystyrene substrates. Although the described technique and the use of a patterned substrate has applicability in the context of many different cell types neurons and cancer cells are among the cell types of relevance. The patterned substrate can, for instance be used for selective attachment and growth of human umbilical vein endothelial cells, MDA-MB-231 human breast cancer cells, NIH 3T3 mouse fibroblasts, and primary rat cortical neurons. The dry-patterned substrate provides particular advantages when implemented in a microfluidic device configured to fluidically isolate different portions of a cell. When implemented in this way the cells can be maintained for a period of time and confined to the cell-adhesive region. For instance, in cases using rat neurons for purposes of test, the neurons can be maintained for a number of days and the neurons' somas and processes were confined to the cell-adhesive region. The method described offers a convenient way of micropatterning biomaterials for selective attachment of cells on the substrates, and enables culturing of patterned cells inside microfluidic devices for a number of biological research applications where cells need to be exposed to well-controlled fluidic microenvironment.

For most applications in cell biology, micropatterns of surface proteins in the range of 10-100 μm are adequate for cell adhesion and growth. Patterning methods based on soft lithography such as microcontact printing (μCP) and micromolding in capillaries (MIMIC) can routinely produce pattern sizes in ˜1 μm, but yield fragile monolayer modified surfaces. These surfaces are not compatible with microfluidic device fabrication steps that require exposure to reactive oxygen plasma for assembly (fluidic bonding). Although direct patterning of biologically active molecules using soft lithographic techniques has many advantages, it is difficult to combine it with microfluidic devices due to the following; (1) residual organic solvent after patterning, (2) oxidation of biologically modified regions during reactive plasma treatment, and (3) contamination of device. Recently, Tourovskaia et al. have reported a method for generating cellular patterns on substrates coated with interpenetrating polymeric network (IPN) of poly(acrylamide) and poly(ethyleneglycol) film by patterned etching with oxygen plasma. Although this method is successful in generating cell-adhesive areas by removing cell non-adhesive IPN film (19 nm thick), it required approximately 15 min of repeated exposure to plasma. This limited the smallest feature to 15 μm because the elastomeric mask is heated and distorted during plasma treatment. For purposes of application to a multi-chamber microfluidic device such distortion is problematic.

To overcome this and other such problems this invention created a new techniques to enables patterned cell culture inside microfluidic devices. Patterning, binding and sterilization steps are carried out in a one or more steps to yield a microfluidic device with patterned surface properties. The procedure uses a small elastomeric poly(dimethylsiloxane) (PDMS) patterning piece with embossed surface features to define the cell-adhesive/non-adhesive areas and a separate microfluidic PDMS piece with microchannels to complete the microfluidic device. Although the invention is not to be limited to such measures, the minimum feature size test in our laboratory is 3 μm, comparable to μCP. Several mammalian cell types including primary rat cortical neurons, human umbilical vein endothelial cells (HUVEC), MDA-MB-231 breast cancer cells, and NIH 3T3 mouse fibroblasts were successfully cultured on the patterned surfaces. Viability for patterned neurons inside the microfluidic devices can be demonstrated for up to 6 DIV although longer periods of time may be achieved, particularly for different cells type which are contemplated as being with the scope of the invention. Viability of cells in the devices depends upon the cell type chosen and the microenvironment created, both of which may be varied as per decisions made by the user of the microfluidic device.

Substrate Preparation

Clean glass coverslips (Corning, N.Y.) should be coated with sterile aqueous solution of 0.5 mg mL⁻¹ poly-L-lysine (PLL, MW. 70,000-150,000, Sigma, Mo.) according to published procedures (See e.g., G. Banker and K. Golsin, Culturing Nerve Cells, The MIT Press, Carnbridge, 2nd ed., 1998, ch. 13). Coated cover slips should be thoroughly rinsed in sterile water for approximately 5 times and air-dried prior to use. Patterned PLL is visualized by conjugating fluorescein isothiocyanate (FITC, Molecular Probes, Oreg.) to PLL via —NH₂ groups. Fluorescence microscopy or other acceptable substitutes can be used to image FITC-conjugated PLL. Sterile bacteriological polystyrene (PS) Petri dish (Fisher, Pa.) are kept sterile and used as received. All coating procedures should generally be performed inside a laminar flow hood or other sterile environment to minimize contamination.

Surface Micropatterning

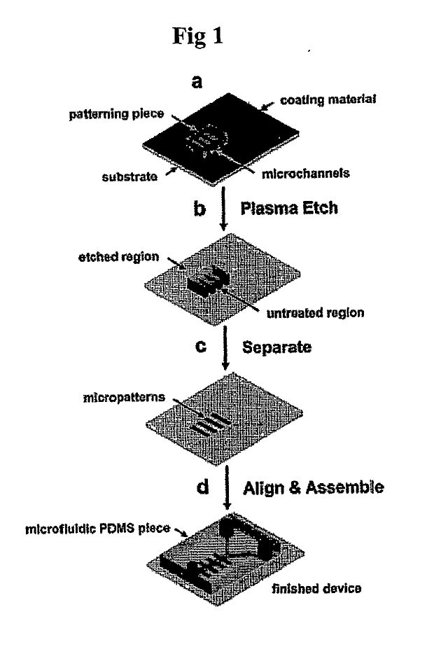

FIG. 1 shows a detailed schematic outline of the procedure for micropatterning cells inside a microfluidic device using cell-adhesive or non-adhesive substrates. This method uses reactive oxygen plasma treatment to accomplish both surface patterning and activation of the substrate and PDMS for assembling the microfluidic device. (a) A small patterning PDMS piece with embossed surface pattern is placed on a substrate that is coated with a thin film. (b) Exposure to reactive oxygen plasma selectively removes material in regions where the patterning piece does not contact the substrate. For instance a PDMS (Sylgard 184, Dow Corning, Mich.) patterning piece for dry-patterning may be fabricated by casting the prepolymer against a silicon wafer master and curing for 15 h at 70° C. A small, PDMS patterning piece, having desired surface embossed patterns can then be placed on the PLL coated glass substrate or PS Petri dish, pressed with a stainless steel weight (100 g cm²), and exposed to reactive oxygen plasma using a plasma cleaner, PDC 001 (30 W, 200-600 mTorr, Harrick Scientific, N.Y.) for 5 s-10 min. (c) After the patterning PDMS piece is removed, well-defined surface micropatterns of cell-adhesive or non-adhesive materials that can be used for selective cell attachment and growth. (d) A microfluidic PDMS piece with microchannel is aligned and bonded to the patterned substrate. The finished device can be used to culture patterned cells inside a microfluidic device.

As briefly mentioned above and now to be described in more detail, a first a substrate is coated with a thin film of either cell-adhesive or non-adhesive material. We have used PLL, collagen, and other extracellular matrix (ECM) proteins (cell-adhesive) as well as untreated PS and other cell non-adhesive substrates. Poly-L-lysine and collagen are commonly used ECM coating materials in cell biology and are suitable for this purpose. Both microcontact printing (μCP) and micromolding in capillaries (MIMIC) can be used to create micropatterns on the substrates and obtained cellular patterns. One important drawback for the above two methods when used for obtaining patterned cells inside microfluidic devices is that reliable seal (bonding) between the substrate and the PDMS microfluidic device is sometimes difficult to obtained. In order for PDMS to bond to a substrate irreversibly, clean surfaces are essential. Surfaces that have been previously modified with SAMs or other organic monolayers and proteins cannot reliably be used to bond irreversibly with PDMS. Although those samples may still work when PDMS is placed in conformal contact, there is higher rate of failure and the device can not be pressurized.

In making a device configured in accordance with one embodiment of the invention, for instance, two different pieces of PDMS can be prepared for this experiment, a first patterning piece (e.g., 4×4 mm²) having to generate the surface pattern and a larger microfluidic piece (e.g., 20×30 mm²) with embedded microchannels for the microfluidic device. The patterning PDMS piece is placed on a large substrate (e.g., FIG. 1, part a) and the entire assembly then placed inside a vacuum plasma chamber (e.g., FIG. 1, part b). A small weight (100 g cm⁻²) can be placed on top of the patterning piece to enhance contact with the substrate and to prevent movement during evacuation of the vacuum chamber. The microfluidic PDMS piece can also be placed in the plasma chamber to activate it for bonding. After approximately 60 s of exposure to oxygen plasma, the coated areas not in contact with the patterning piece are completely etched away. This leaves a pattern of cell-adhesive and non-adhesive areas for selective attachment of cells. Because the PLL and collagen coatings form a thin coating (PLL thickness is ˜1 nm, measured with an ellipsometer, comparable to a monolayer of polyelectrolyte film), short plasma treatment of 60 s is adequate to completely etch away the coating. For cell non-adhesive substrate like PS, this short exposure to oxygen plasma converts it to oxidized PS (PS-ox) which is hydrophilic and adhesive to cells. Therefore, for cell-adhesive substrates, the region where the patterning piece contact the substrate is protected from the etching plasma and yields a positive cellular pattern that is identical to the pattern on the patterning piece (e.g., FIG. 1, part c). In contrast, a “negative cellular pattern is obtained for a cell non-adhesive substrate after plasma treatment.

After a small area of patterned cell-adhesive and non-adhesive is defined on the substrate, the microfluidic PDMS piece can be visually aligned and bonded to complete the device. Because the patterning piece covers a small area, the etched area outside the pattern is activated and can be used to bond the substrate with a microfluidic PDMS piece. (e.g., FIG. 1, part d) The completed device can now be used to culture cells on a micropatterned surface that is enclosed within the microfluidic channels. Although a wide variety of substrates can be patterned using the method described in this work, there are some limitations for ECM proteins that can denature and lose their biological activities when dried. However, these limitations can be overcome by using both cell-adhesive and non-adhesive materials. For example, a substrate can be first coated with cell non-adhesive material (bovine serum albumin, alkylsilane and poly(ethyleneglycol)) and the area exposed to oxygen plasma can be backfilled with a fragile ECM protein after assembling the substrate with the microfluidic PDMS piece.

Generally with respect to patterning conditions, dense and small features take longer time when compared to large, sparse patterns. Etch times and other experimental conditions are adjusted depending on the equipment used and some variation is well within the scope and spirit of the invention described herein in the context of an example. In general, for bonding application, short treatment times at medium power is used (200 mTorr, 10 W, 60 s). Longer plasma treatments at high power result in over-oxidized PDMS surface that do not bond to other surfaces. The plasma treatment times reported in the manuscript (5-120 s) were optimized for PDMS bonding using a basic plasma chamber. For applications that require longer time to etch surface patterns, the plasma-exposure may be divided in two stages that include etching (substrate and masking PDMS) followed by bonding (insert the microfluidic PDMS piece) treatments. For example, if a total of 5 min is needed to etch the substrate, only the substrate is placed in the plasma chamber during the first 3 min followed by substrate and microfluidic PDMS piece in the last 2 min. This approach minimizes plasma exposure for the microfluidic PDMS piece and can yield optimal bonding. It also ensures that the substrate coating is completely removed so that reliable irreversible bonding can be formed.

Fabrication of Microfluidic Cell Culture Device

A separate PDMS piece is typically prepared for microfluidic device fabrication. Although there are different techniques for fabricating the various types of microfluidic devices one or more embodiments of the invention may utilize, the microfluidic cell culture device can, for example, be fabricated in PDMS using rapid prototyping and soft lithography. Using this fabrication technique the master for the neuronal culture device is fabricated by patterning two layers of photoresist. A first layer of photoresist, 3 μm thick is obtained by spinning SU-85 negative photoresist at 3,500 rpm for 60 s. A 20,000 dpi high-resolution printer provides a means to generate the first transparency mask to create the microchannels (10 μm wide and spaced 50 μm). The transparency mask is used to pattern the SU-85 photoresist. Second layer of thick photoresist (100 μm) can be spun on top of patterned 3 μm features. SU-8 50 is used as a second layer and spun at 900 rpm for 60 s. Separate, second mask can used to create the chamber areas aligned to the first pattern. After development, the wafer may be placed in a clean Petri dish and mixture of PDMS-prepolymer and catalyst (10:1 ratio) is poured over the maser. The Petri dish containing the wafer is placed in an oven for 15 h at 70° C. Positive replica with embossed microchannels can then be fabricated by replica-molding PDMS against the master. The inlets and outlets for the fluids may be punched out using sharpened blunt-tip needles or other sharp or blunt objects. The surface of the PDMS replica and a coated glass substrate are activated with reactive oxygen plasma and brought together by visual alignment immediately after activation to form an irreversible seal. Other aspects of the microfluidic device described herein are described in U.S. patent application Ser. No. 10/605,537 entitled “MICROFLUIDIC DEVICE FOR NEUROSCIENCE RESEARCH” and filed on Oct. 6^{th}, 2003 which is incorporated herein by reference.

Sterilization and Fluidic Bonding.

An important issue in using microfluidic devices for cell culture involves sterilizing the assembled device. Sterilizing processes such as UV exposure and autoclaving may not be used for microfluidic devices because substrates were coated with biomaterials. The plasma etching/sterilization equipment is kept free of problematic contamination and should, for instance be placed inside a biological safety cabinet or some other clean environment to avoid potential contamination problems. All process steps should typically be carried out in sterile conditions. Performing device assembly inside a biosafety cabinet has the additional benefit of reducing particulate contamination. When transporting substrates and materials, they should also be kept inside sterile containers. The process of bonding microfluidic PDMS piece to a substrate using oxygen plasma treatment can also serve as a sterilization step. The plasma treatment time is typically optimized and can be varied for PLL patterning such that this step can be used for sterilization as well as bonding.

One or more embodiments of the invention involve the use of different mammalian cell types grown in the patterned micro-fluidic device described herein.

Mammalian Cell Culture

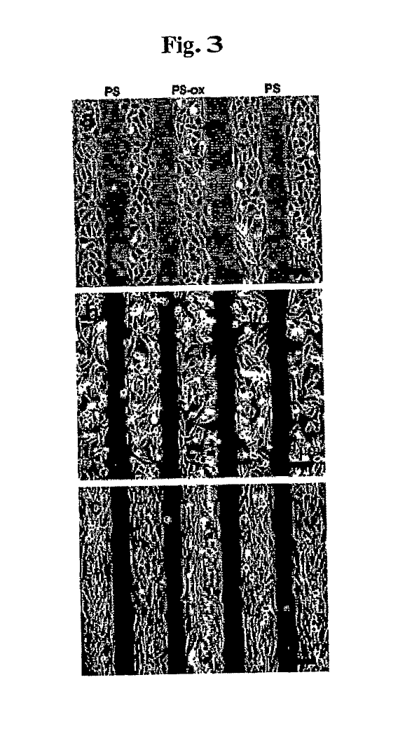

FIG. 3 shows patterned HUVEC, MDA-MB-231 human breast cancer cells and NIH 3T3 mouse fibroblasts cultured for 548 h on patterned Petri dishes grown in accordance with one or more embodiments of the invention. In growth tests conducted, the metastatic human breast cancer cell line MDA-MB 231 (ATTC, MD) can be cultured in Leibovitz's L-15 medium (Invitrogen, CA) supplemented with 10% FCS. Primary HUVEC were cultured in M199 medium supplemented with 10% FCS, heparin (5 U μL⁻¹), 1% endothelial growth factor (Sigma, Mo.), and antibiotics. The NIH 3T3 mouse fibroblasts were cultured in DMEM containing 10% FCS. Dissociated cells were plated on the patterned substrates at approximate density of 5×10³−1×10⁵ cells cm⁻², and cultured in a humidified incubator at 37° C. Readers should note that although specific cancer cells were used for purposes of describing the process stated herein other cells types have successfully been grown and the invention is by no means limited to the specific cells types stated herein as other cells are fully contemplated as being within the scope and spirit of the invention.

To grown mammalian cells using the device described herein the cells users may start with non-tissue culture grade Petri dishes and generated patterned cell-adhesive areas on them. Non-tissue culture grade Petri dishes made of PS are usually used for suspension cultures while tissue culture grade PS dishes are used for culturing adherent cells. Physico-chemical properties of oxidized PS surfaces are very similar to tissue culture dishes that are commercially available. Treatments of non-tissue culture grade PS Petri dishes to reactive oxygen plasma can turn the normally hydrophobic PS surfaces into hydrophilic surfaces, allowing cells to adhere and spread. The effect of patterned exposure of cell non-adhesive PS Petri dish to oxygen plasma is clearly demonstrated by the patterned cells shown in FIG. 3. The cells exhibited preferential attachment and growth on 120 μm wide oxidized areas, whereas the untreated areas (areas where patterning piece contacted the PS substrate) were devoid of cells. All three cell types have similar morphologies to those cultured on control tissue culture grade Petri dishes. Short exposure (2 min) to reactive oxygen plasma effectively changed the PS surface properties and made it cell-adhesive. Longer plasma etching up to 5 min showed similar results. Occasionally, some cells were able to weakly adhere on untreated PS region, but those cells did not spread and remained round, eventually detaching from surface after a day.

The images show in FIG. 3 show (a) HUVEC cultured for 5 h, (b) MDA-MB-231 breast cancer cells cultured for 36 h, and (c) NIH 3T3 mouse fibroblasts cultured for 48 h on the modified oxidized PS patterns. A small patterning PDMS piece (10×10 mm²) with channels (120 μm wide, separated by 80 μm spacing and 100 μm deep) can be placed on non-tissue culture grade PS Petri dish. The entire assembly can be exposed to oxygen plasma for 2 min. The regions exposed to plasma (120 μm wide channels) were oxidized (PS-ox) and became hydrophilic. When cells are added to the modified Petri dish, they preferentially attached, spread, and proliferated on hydrophilic areas exposed to oxygen plasma.

Neuronal Cell Culture

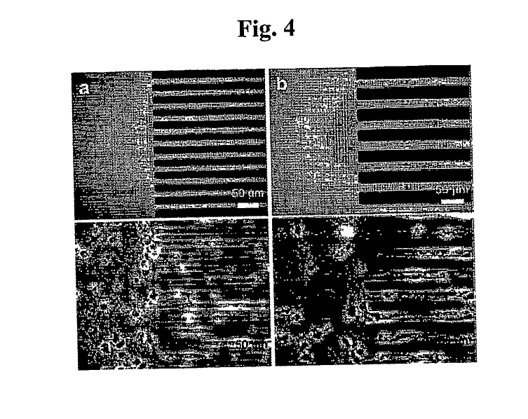

FIG. 4 shows rat cortical neurons grown using the patterned microfluidic device configured in accordance with one or more embodiments of the invention. FIG. 4 is an example that represents and specifically shows the compatibility of the patterning method with microfluidic device fabrication. In this instance, patterned neurons were maintained inside a microfluidic device for 6 DIV. Readers should note however that this viability time varies depending upon cell type and that more or less time is feasible based on the microenvironment created. Primary rat cortical neurons are used here because they are one of the most difficult cells to culture as they are extremely sensitivity to their culture conditions. As such substantial improvements with other cell types are expected. Successful demonstration of the approach with the neurons strongly confirms the validity of the method and indicates that the approach will work with other cell types.

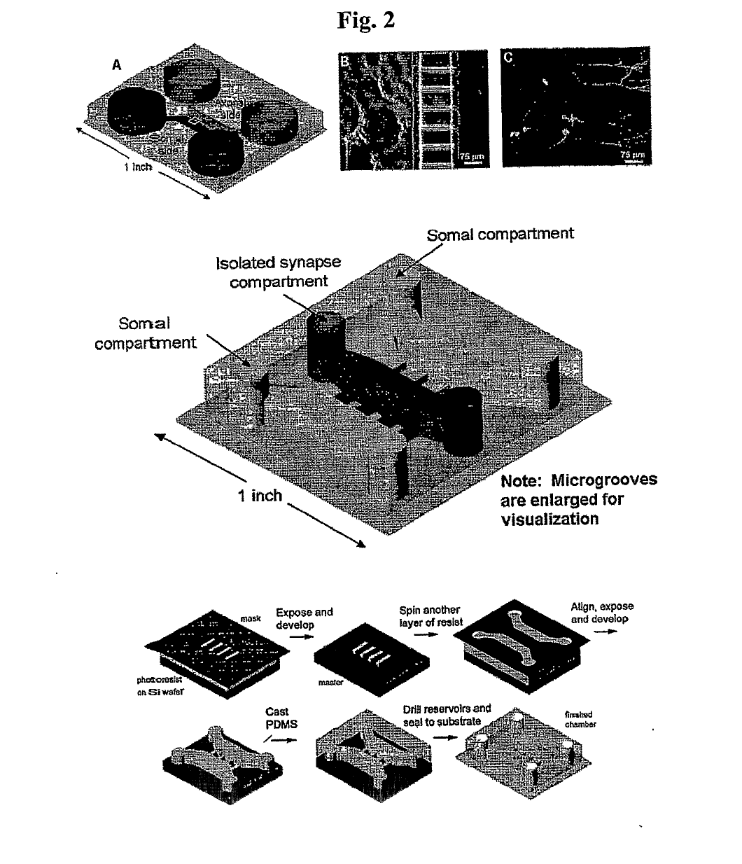

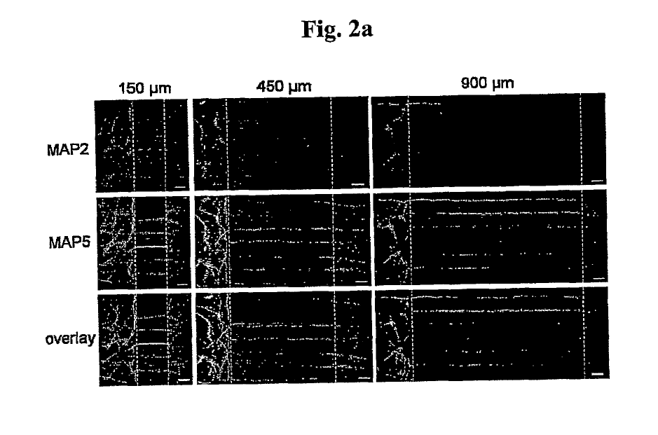

A compartmented microfluidic neuronal culture device can be fabricated in PDMS to achieve fluidically isolated microenvironments for somas and neurites. FIG. 2 shows the schematic of a compartmented microfluidic neuronal culture device that can be assembled on a PLL micropatterned glass substrate. A photograph of neurons grow in accordance with this device is depicted in FIG. 2a. Three fluidically isolated compartments (approximately 1 mm wide, 7 mm long and 100 μm high—sizes may vary) are separated by an approximately 100 μm wide barriers as shown. The barriers have embedded microgrooves (3 μm high and 10 μm wide) that allow neurites to grow across the barriers from somal to neuritic compartments. The compartments are connected to each other with a number of microgrooves (e.g., 3 Mm high and 10 μm wide—although the specific sizes may vary per groove or across all the grooves). Each compartment fluidically isolates different neuron regions (soma and neurites were separated from each other). The size of the microgrooves is sufficiently small that unattached neurons do not pass through the microgrooves to the adjoining compartments during loading. This design simplifies the loading process and allows selective placement of neurons in one compartment. There are large holes at the end of the compartments that serve as cell loading inlets and medium reservoirs for nutrient and gas exchange. The volume in each compartment (without the reservoirs) is less than 1 μL. In comparison, the combined reservoirs for each compartment can hold up to 200 μL. By having such small culture volumes, reagent amounts can be significantly reduced compared to traditional culturing methods. In addition to isolating somas from their processes, users are able to pattern the growth of neurites on the substrate inside the microfluidic device. In one embodiment of the invention the microgrooves in the barrier are aligned with micropatterned PLL lines that guide the growth of neuritic processes as shown in FIG. 2.

Micropatterning of the cells and their processes facilitated identification of cells and improves visualization of results. For example, in a random culture on a tissue culture dish, due to the entangled network of dendrites and axons, it is difficult to determine the respective soma for a particular process. Fluorescence micrographs of live, calcein AM stained cells follow patterned PLL, allowing readily identification of cells. This photograph shown in FIG. 4 can be taken after 6 DIV of culturing neurons inside the microfluidic device. The neurons are initially loaded into the two outer compartments and allowed to send out processes. Two thick black lines are the 100 μm barriers that separate the compartments. As shown, the bright spots indicate that somas are present in the outer two compartments but not the middle. The middle compartment contains neuritic processes that were sent out from the opposite compartments. FIG. 4, part c shows a series of time-lapse images taken of a pair of processes in the middle compartment projecting from two different neurons in opposite compartments of the device. After approximately 3 to 4 days of growth, neurites from the somal compartment (outer compartments) extend into the neuritic compartment (middle compartment). After 6 DIV, neurites meet in the middle compartment. These micrographs illustrate that the substrate patterning methods can be combined with microfluidic devices to generate controlled microenvironments for different regions of neurons.

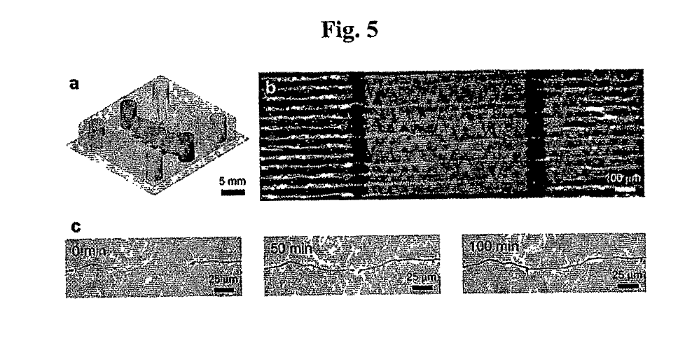

Primary cultures of E18 rat cortical neurons were prepared as described previously. As show in FIG. 5 Dissociated cells were plated on the PLL treated substrates and in the microfluidic channels at a density of approximately 3×10⁴ cells cm². The cells were cultured in the neurobasal medium supplemented with 2% B27 and 0.25% GlutaMAX in a humidified incubator (Thermo Form a, OH) at 37° C. with 5% CO₂. Live neurons were stained with 1 μM calcein AM (Molecular Probes, Oreg.) in the culture medium. As FIG. 4 and FIG. 5 shows rat cortical neurons are able to be successfully grown in the microfluidic device. For instance, part (b) of FIG. 4 shows fluorescence micrograph of rat cortical neurons cultured on PLL patterned glass substrate (25 μm wide lines with 25 μm spacing) inside a compartmented microfluidic neuronal culture device. Neurons were plated into the outer two compartments and cultured for 6 DIV. Live cells were brightly stained by a viability dye, calcein AM. (c) A series of time-lapse images were taken at the middle compartment after 6 DIV of culture. The images show two different processes growing toward each other while respective somas were located in the two outer compartments. The processes follow and remain within the PLL pattern as they extend and eventually meet.

Imaging/Microscopy

One benefit provided by use of the microfluidic devices is that users can conduct imaging throughout an experiment and hence obtain data that allows the user to acertain the effectiveness of a particular compound as opposed to another. For instance, in addition to regular photographs or video it is possible to take phase-contrast and epifluorescent images using equipment such as an inverted microscope, Nikon TE 300, CoolSNAPcf CCD camera (Roper Scientific, Ariz.), and MetaMorph (Universal Imaging, Pa.). Although any mechanism for accomplishing the same will suffice, Lambda DG-4 (Spectra Services, N.Y.) can be used as an excitation light source which can be controlled by MetaMorph For long term culture on the microscope stage, time-lapse images can be were acquired every 5 min for 12 h. Such imaging is useful for purposes of conducting evaluation into an experiment and/or learning and evaluating the results of a particular solution applied to one or more regions of a cell.

Centrifugal Cell Positioning

One embodiment of the invention allows for cells within the microfluidic device to be positioned through the use of centrifugal force. External forces (centrifugal, hydrodynamic, and gravitational forces), when applied to micrometer-scale objects (i.e. cells) inside microfluidic device, can effectively transport and position cells in preferred locations inside a microfluidic channel. Except for centrifugal force-based positioning that can be used with any microfluidic channels, hydrodynamic and gravitational force-based positioning yield reproducible and optimum results when implemented with a microfluidic “module” that contains a barrier with embedded microgrooves. Primary rat cortical neurons, metastatic human breast cancer cells MDA-MB-231, NIH 3T3 mouse fibroblasts, and human umbilical vein endothelial cells (HUVECs) are compatible with the positioning process and hence used herein for purposes of example; the invention however is not limited specifically to the exemplary cell type. After positioning, cells attached, proliferated and migrated like control cells that were cultured on tissue culture dishes. To demonstrate a practical application of the method, cells were placed in a single row along a wall using centrifugal force and gravitational force. Cell positioning allows that all cells get exposed to the same level of chemoattractant at the start of the experiment helping standardize cellular response.

The ability to pattern and control placement of cells on the micrometer-scale is important for applications in tissue engineering, biosensors, and for investigating fundamental cell biology questions. A general approach to patterning utilizes photolithography and soft lithography to modify the surface properties (adhesive and non-adhesive regions) followed by selective cell attachment in adhesive regions. Although patterning techniques such as microcontact printing (μCP) and micromolding in capillaries (MIMIC) are one approach and have applicability across an extensive range of applications, their use with microfluidic devices have been limited due to compatibility issues also solved herein.

Microfluidics-based cell culture has advantages over conventional tissue culture dish-type cultures as it offers precise control of cellular microenvironments with an added advantage of significantly reduced reagent consumption. When cells are detached from their culture flask and loaded into microfluidic devices, there is a short period of time during which they settle down and attach to the substrates. The approach used in one or more embodiments of the invention takes advantage of this time interval by applying a combination of centrifugal, hydrodynamic, and gravitational forces, to cells while they are in suspension. These forces, generally ineffective in the macro-scale but exert significant effect in micro-scale, can effectively transport and position cells in preferred locations inside a microfluidic channel.

The approach described here to positioning cells within microfluidic devices can be implemented without special equipments or additional fabrication steps (i.e. microelectrodes). The cells are able to attach, proliferate, and migrate like control cells that were cultured on tissue culture dishes. As an example, primary rat cortical neurons were successfully patterned on stripes of adhesive surface with somas positioned on one side of the microchannel. A practical application of cell positioning is demonstrated for chemotaxis assays.

Substrate Preparation

Glass coverslips (24×40 mm², No. 1) area obtained and cleaned by immersion in 2% of aqueous Micro-90™ cleaning solution (Cole Parmer Instrument Co., IL) at room temperature for 24 h and sonicated in cleaning solution for 5 min. The cleaned glass coverslips were repeatedly rinsed in deionized (DI) water (5 times) and dried before use.

Fabrication of Microfluidic Cell Culture Device

The microfluidic cell culture device can be fabricated in PDMS using rapid prototyping and soft lithography following procedures described herein. Positive replica with embossed microchannels can be fabricated by replica-molding of PDMS against the master. The surfaces of PDMS replica and glass substrates were activated with reactive oxygen plasma and brought together immediately to form an irreversible seal. For neuronal cultures, the substrates were coated with poly-L-lysine (PLL) by immersing in sterile aqueous PLL solution (0.5 mg ml⁻¹, MW=70,000-150,000, Sigma, Mo.) for 15 h at room temperature and rinsed in DI water (3 times) before use. For breast cancer cell migration assay, the substrates were coated with 2 μg mL⁻¹ of collagen type IV (Sigma, Mo.) for 1 h at room temperature and blocked with 2% BSA in Leibovitz's L-15 medium for 1 h at 37° C. before use.

Positioning Cells

All steps described in connection with cell positioning should be performed in an environment to minimize contamination. For instance, in one embodiment of the invention steps are performed inside a laminar flow bench to minimize contamination. Cell suspensions (25 μL) are typically introduced into the middle main channel and an external force applied relatively soon after the introduction. For centrifugal force-based positioning, all inlet and outlet holes are typically sealed with adhesive tapes before placing the device on a spinner. The device is to be fixed at a given distance (0-5 cm) from the axis of rotation and spun at 500-4,000 rpm for 30-300 sec. For gravitational force-based positioning, devices were tilted for 10-20 min after cell loading. To use hydrodynamic force, one of the side channel's reservoirs can be kept at higher level compared to the main channel. For example, left reservoir can be filler with 200 μL of medium before loading the cell suspension (25 μL) into the middle reservoir. Right side channel can be intentionally left without fluid. Similar results were obtained by applying weak suction to the right channel. Short aspiration with house vacuum can be adequate to move the cells. Above methods (gravitational, hydrodynamic, and aspiration) can be used individually or in combination for reproducible results.

The NIH 3T3 mouse fibroblasts were cultured in DMEM containing 10% fetal calf serum (FCS). The metastatic human breast cancer cell line MDA-MB 231 (ATTC, MD) can be cultured in Leibovitz's L-15 medium (Invitrogen, CA) supplemented with 10% FCS. HUVECs were cultured in endothelial cell basal medium 2 (EBM-2, Clonetics, Calif.) supplemented with FCS, hydrocortisone, hFGF-B, VEGF, R3-IGF-1, ascorbic acid, heparin, hEGF, and GA-1000. Primary cultures of E18 rat cortical neurons were prepared as described previously. The neurons were cultured in the neurobasal medium supplemented with 2% B27 and 0.25% GlutaMAX. Dissociated cells were plated in the microfluidic channels at an approximate density of 1-6K 10⁶ cells mL⁻¹, and cultured in a humidified incubator at 37° C. Live cells were stained with 1 μM calcein AM (Molecular Probes, Oreg.) in culture medium. Plasma-based dry etching method can be used to patterned culture of neurons on PLL stripes.

Cell Migration

Cell migration experiments were performed with a microfluidic chemotaxis chamber (MCC) following previously reported procedure. 28 Metastatic breast cancer cells, MDA-MB 231, were serum starved overnight in 0.2% BSA in Leibovitz's L-15 medium before use. Cells were detached from the culture flask using cell dissociation buffer (Invitrogen, CA), can behed and resuspended in growth medium, and then filtered through a nylon strainer (40 μm) to obtain a single cell suspension. Cells were loaded into the channel using a micropipette. Epidermal growth factor (EGF) solution can be prepared in Leibovitz's L-15 medium with 0.2% BSA containing 1 μM of FITC-dextran (MW. 9.5 kDa, Sigma, Mo.) as an indicator for EGF gradient. Soluble EGF gradient can be generated by continuous infusion of 50 ng mL⁻¹ of EGF and medium into two separate inlets into MCC.

Design of Microfluidic Device

The microfluidic device used to position cells is more fully described above and is made up of three separate channels separated by physical barriers that have embedded microgrooves. Each barrier has 120 embedded microgrooves (width=10 μm, height=3 μm, length=100 μm) fluidically connect the channels. The micrometer-size grooves is sufficiently small that cells (assuming 10-15 μm sphere in suspension) do not pass over to the adjoining channels but fluid can be moved across the barrier with significant resistance. The cells were placed in the middle main channel (width=800 μm, height=100 μm, length=7 mm) for patterning while applying aspiration or hydrodynamic focusing either of the two side channels. Schematic illustration of positioning cells using external forces is shown in FIG. 1b. When cells are detached from their culture flask and loaded into the microfluidic device as individual cells, it takes a few minutes to settle down and attach. Applying combinations of centrifugal, hydrodynamic, and gravitational forces before the cells settle down, it is possible to position cells in preferred region of the channel.

Centrifugal Force-Based Cell Positioning

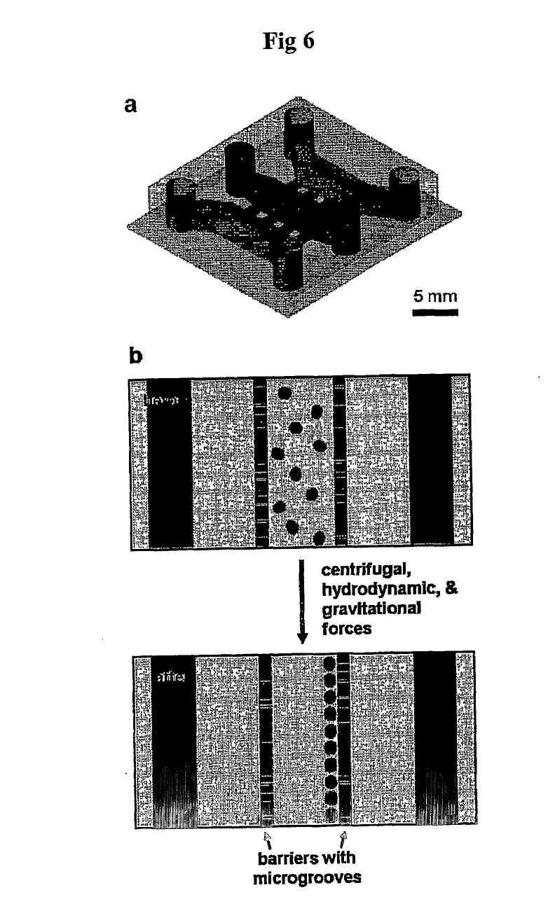

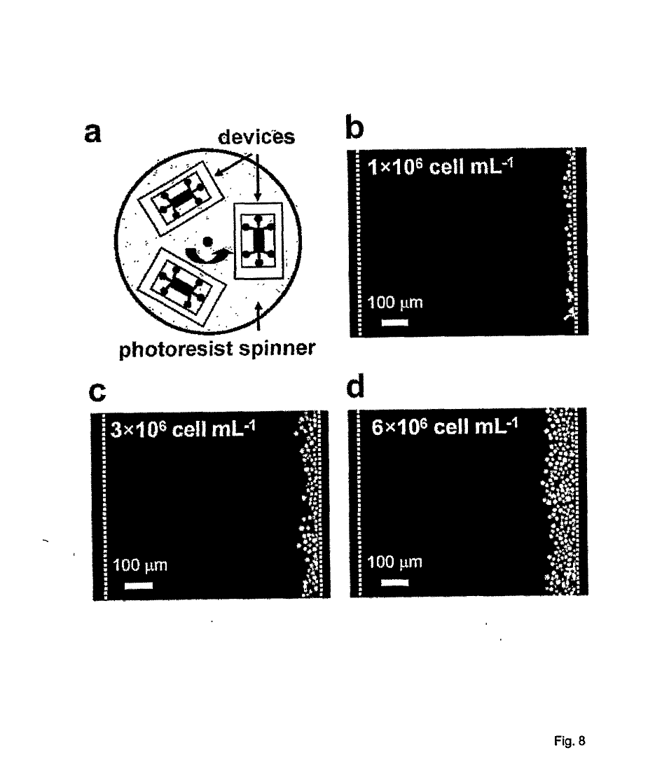

Centrifugal force is used ubiquitously in laboratories to separate and purify cells and biomolecules. Embodiments of the invention use centrifugal force to move and position cells inside microchannels. FIG. 6 shows a schematic of the experimental step and the results. A photoresist spinner can be used to generate the centrifugal force in this work. Other instruments such as laboratory centrifuge and other similar equipments can also be used. Since the centrifugal force exerted on the cells in this work is smaller (−20-25 g) than those used to pellet cells using a laboratory centrifuge (−220 g), the viability of the positioned cells were not adversely affected. An important advantage of this method is that the number density of positioned cells can be controlled by adjusting the density of the cell suspension. The numbers in FIGS. 8b, c, and d indicate the density of starting cell suspensions.

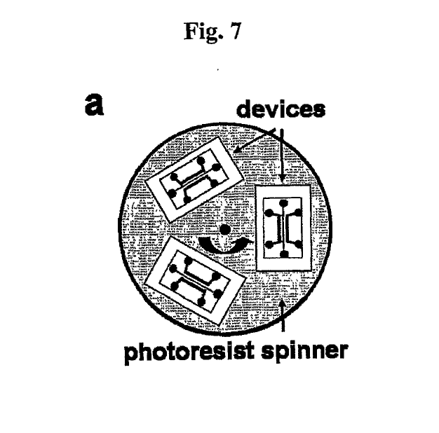

The spinner can be placed inside a laminar flow bench and all steps were carried out in sterile conditions. To subject the cells to centrifugal force, assembled microfluidic devices were placed on the spinner and cell suspension can be loaded into the main channel. Microfluidic devices were placed on the spinner such that the main channel can be parallel to the direction of rotation while taking into account of approximate distance to the axis of rotation as shown in FIG. 7. The centrifugal force V) experienced by the cells in a rotating platform is

f=mrω² (1)

where m is mass of cells, r is distance from axis of rotation and ω is rotational speed.

Although various rotation speed that are not destructive on the cells to be positioned can be used and are contemplated as being part of the invention. In one embodiment of the invention the Distance from axis of rotation varies from 0 to 5 cm at 2,000 rpm (209 rad s⁻¹) and the rotational speed varies from (500-4,000 rpm) at fixed distance of 5 mm. For example, when pelleting a suspension of cells using conventional centrifuge, relative centrifugal field (RCF) of 220 g (1,000 rpm at 20 cm from center) is experienced by the cells. Under our experimental conditions we found that RCF of −20-25 g can be optimum for most cells (2,000 rpm at 5 mm from center). At lower RCF, most cells were randomly distributed with a small fraction positioned near the barrier. At higher RCF (>90 g), several problems were encountered. First, cells deformed and squeezed into the microgrooves and the gap between the substrate and PDMS mold, affecting their viability. Second, for cell types that settle and attach within short duration (i.e. neurons), significant portion of the attached cells were lysed due to shear during spinning.

Fluorescence micrographs of NIH 3T3 fibroblasts positioned inside microfluidic channels using centrifugal force are shown in FIGS. 2b, c, and d. Suspension of fibroblasts (20 μL of cell suspension with different cell densities) can be introduced into the microfluidic device and the device can be spun at 22 g (2,000 rpm. at 5 mm from the center of rotation) for 2 min. The cells were allowed to attach for 20 minutes and stained with a viability marker, calcein AM. As shown by the brightly stained cells in the figure, all cells were live and viable. Densities of plated cells are roughly proportional to those of starting density of cell suspension. The density of cell suspension could be adjusted to yield a single row of cells to a thick band of cells within the microchannel.

For practical applications, air bubbles need to be avoided. After filling the microchannels with cell suspension, care can be taken to completely seal the fluidic inlets and outlets with adhesive tapes or other objects. If the holes were not completely sealed, bubbles formed and passed through the channels, lysing and removing the attached cells. There are some limitations with the cell types that can be used with centrifugal force-based positioning. Cells that attach firmly and rapidly, i.e. neutrophils, are difficult to work with. Although we have successfully worked with primary rat cortical neurons, the experiments need to be carried out swiftly as they attach on PLL coated surfaces within few minutes. In contrast, fibroblasts, cancer cells and HUVECs were easier to handle as they were robust and withstood the stress of handling.

Experimental conditions will need to be optimized depending on the particular cell type (size, density, and adhesion receptor expression), cell-surface adhesion, microchannel dimension, and media composition (Ca and Mg free media to minimize integrin-mediated adhesion), and other experimental variables.

Cell Positioning using Combined Forces

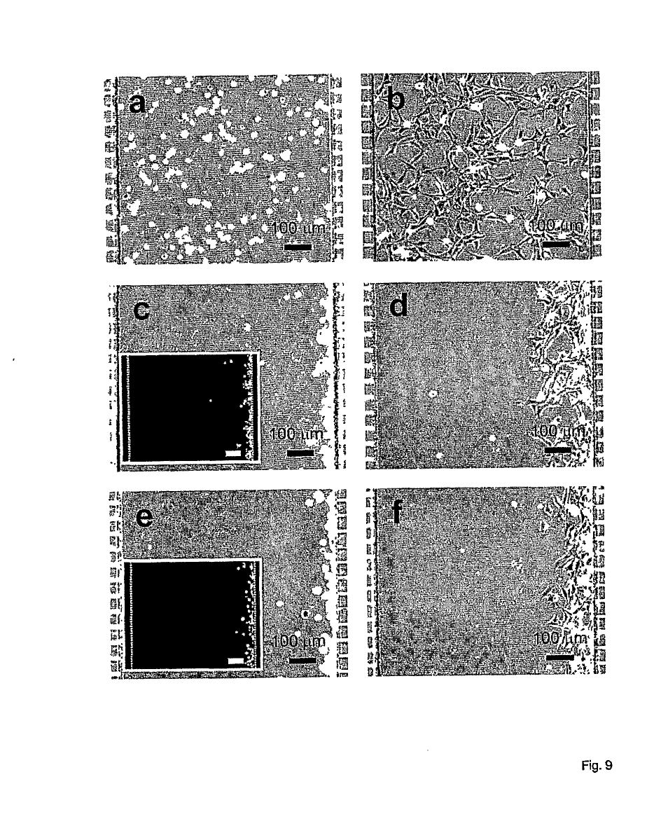

In contrast to centrifugal force-based cell placement which can use any type of microfluidic channel design, the results described below require a distinctive barrier design to work effectively. In order to effectively use hydrodynamic force and aspiration to position the cells, an array of embedded channels are required. This “module” with embedded microgroove barrier (dimension of microgroove is 3 μm×10 μm) allows fluidic connection between main channel and two side channels while blocking movement of cell bodies (−10-15 μm sphere in suspension). FIG. 9 shows cell positioning results without any external force (FIGS. 9a and b), combination of gravitational and hydrodynamic forces (FIGS. 9c and d), and combination of hydrodynamic force, gravitational force and aspiration (FIGS. 9e and f).

Cells are randomly attached when introduced into the microfluidic channel without any external force. FIG. 9a shows NIH 3T3 mouse fibroblasts 1 h after loading. It takes approximately 5 min for the cells to settle down (microfluidic channels with 100 μm depth) and attach on the substrate. In comparison, it takes 20-30 min for majority of the cells to settle down and attach on tissue culture dishes or flasks (for 2 mm media level in Petri dish). Application of hydrodynamic force, aspiration, and tilting of the device (gravitational force) while the cells are in suspension shifts the cells toward desired region along the microchannel.

Application of single external force (gravitational, hydrodynamic, or aspiration) offered promising results but were not reproducible (data not shown). To optimize the results, we used a combination of two or more forces to position the cells. FIG. 3c and a show results from combinations of two (gravitational and hydrodynamic) and three (gravitational, hydrodynamic, and aspiration) forces, respectively. Gravitational force can be applied by tiling device between 45-70 degrees from horizontal. To apply hydrodynamic force on the cell suspension, three inlets to the channels were infused with 200 μL of medium (left channel), 25 μL of cell suspension (middle channel), and 0 μL (right channel). The difference in volume resulted in 4 mm difference in height of the reservoir, effectively generating hydrodynamic force that focused the cell suspension stream in the middle main channel against the right barrier. To enhance the effectiveness of cell placement, aspiration can be applied in conjunction with gravitational and hydrodynamic forces. While introducing the cell suspension into the middle main channel, weak suction can be applied from the right-channel.

When combination of two or more forces can be applied, most of the cells were positioned near the right barrier. Inset figures show corresponding fluorescence micrographs of cells stained with calcein AM, indicating that the external forces do not adversely affect viability. In addition, micrographs of fibroblasts taken after 24 h (FIGS. 9d and 9) indicate that the attached cells attached and proliferated like control cells (FIG. 9b). Successful results are obtainable using the techniques and devices described herein with several cell types such as cancer cells, HUVECs, and primary rat cortical neurons.

Positioning of Primary Rat Cortical Neurons

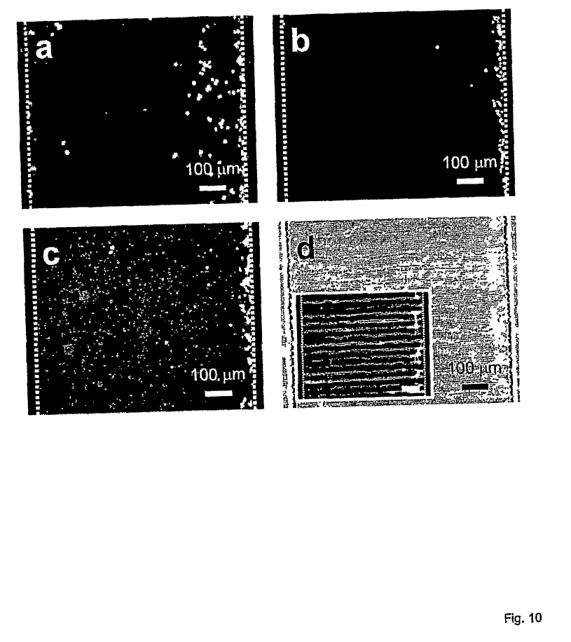

To test the robustness of the methods and applicability to other cell types, we used primary rat cortical neurons that are exceptionally sensitive to culture conditions. The viability of neuronal cultures after positioning is a sensitive indicator of adverse effects on living cells. FIG. 10 shows the fluorescence micrographs of neurons positioned along a wall using; (a) combination of gravitational and hydrodynamic forces, (b) combination of gravitational force, hydrodynamic force and aspiration, and (c) centrifugal force, respectively. Viable cells were stained with calcein AM and are imaged as bright round dots. FIGS. 10a, b, and c show cells that are stained immediately after positioning. FIG. 10d shows phase-contrast micrograph and fluorescence micrograph (inset) of neurons cultured for 7 days in vitro on micropatterned cell adhesive PLL substrate (25 μm wide lines separated by 25 μm) after positioning along a wall with centrifugal force. The neurons were viable and remained healthy for over 7 days. Longer times are feasible in different microenvironments and/or with different cell types.

Furthermore, as shown in FIG. 10d, cells were healthy and most of the processes remained on patterned PLL stripes for over 7 days. As a result, somas are localized close to the right-wall while the axons and dendrites extend across the channel. The channel, 800 μm wide, is large enough such that a portion of the neuron (i.e. soma or a tip of the neuritic processes) can be selectively exposed to a fluid stream containing a chemical (i.e. oxidative stress that can cause degeneration). These results show potential advantage of combining cell positioning with surface patterning methods in basic neuroscience and other forms of cell research.

Cell Positioning in Microfluidic Chemotaxis Chambers (MCC)

Microfluidic devices that can generate precise gradients of chemoattractants have been used in investigating neutrophil and breast cancer cell chemotaxis and can be used for many other forms of cell research where there is a need to apply different microenvironments to different parts of the same cell or cells. Stable soluble gradients produced with MCC allowed detailed quantitative analysis of cell migration data. Because the cells were loaded into the device in random manner, the cells were exposed to different concentrations of chemoattractant. This made it difficult to compare different cells as their starting positions were different. To minimize the variability when comparing cell migration, we used the approaches described in this paper to position the cells along a wall inside MCC such that most of the cells have same “starting position”.

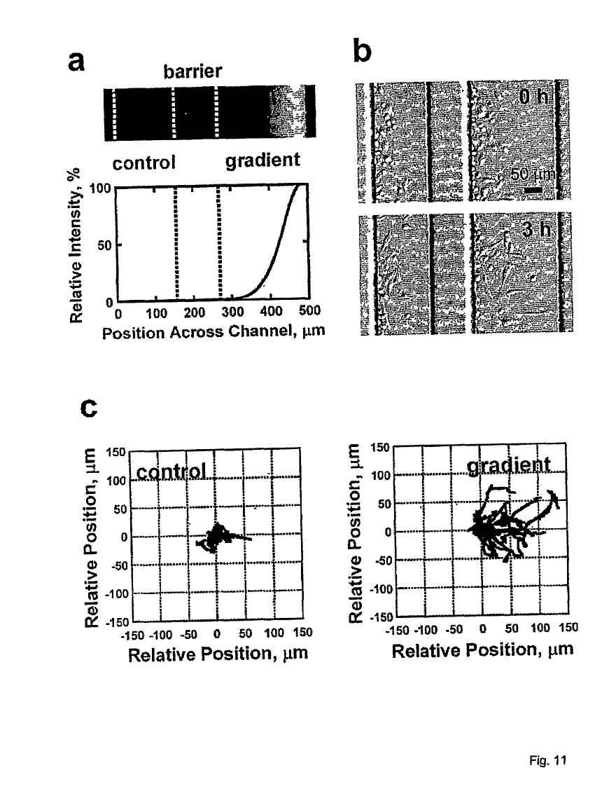

FIG. 11 shows the result from a chemotaxis experiment using human breast cancer cells, MDA-MB 231. It has previously been noted that cells migrated randomly in “control” region of the EGF gradient while migrated in directed manner in steep “gradient” region. Dividing the migration channel into two sub-regions using a physical barrier minimizes this migration. FIG. 11a shows the fluorescence micrograph and the intensity profile of a polynomial gradient (y=ax^{4.2}) ofFITCDextran (MW. 9.5 kDa) in MCC (fluorescent FITC-Dextran with similar molecular weight as EGF, MW. 6.2 kDa, can be used to indirectly verify EGF gradient).

FIG. 11b shows the images from 3 hour experiment of MDA-MB-231 cells migrating in polynomial gradient of 0-50 ng mL⁻¹ EGF. Cells were loaded into MCC and positioned along the left wall by gravitational force. FIG. 11c shows migration tracks of twenty randomly selected cells from each sub-region. In the “control” region, most cells remained within 25 μm from starting position and moved in random directions. In sharp contrast, most of the cells in the “gradient” region migrated over 50 μm and covered longer distances. Although the cells were blocked from moving toward left in both cases, the cells in “control” region exhibited clearly random movement compare to directed migration for the cells in “gradient” region. This difference is also clear in the micrographs shown in FIG. 11b. In contrast to previous works where all cells were randomly located, positioning cells along a wall in MCC makes the comparison between different conditions easier. Further detailed quantitative comparison between randomly loaded cells and positioned cells are in progress.

In summary, we have demonstrated several approaches to positioning mammalian cells (rat cortical neurons, breast cancer cells, NIH 3T3 fibroblasts, and HUVECs) inside microfluidic channels. Cell placement can be achieved by using one or more external forces including centrifugal, hydrodynamic, and gravitational forces in combination with a microfluidic “module”. Positioned cells were viable, and migrated and proliferated like control cells. Use of multiple forces in combination (i.e. hydrodynamic, gravitational and aspiration) yielded reproducible, optimum results in which the cells were successfully isolated on one side of the channel. Optimizing the density of cell suspension can control the number of positioned cells. Furthermore, this microfluidic “module”, a barrier with embedded microgrooves, can be used as a component of other functional microfluidic devices (i.e. as a part of microfluidic chemotaxis chamber). An application of cell positioning is demonstrated for chemotaxis assays. Compared to previous methods where randomly placed cells were exposed to different concentrations of chemoattractants at the start of the experiment, cells can be placed in a single file, providing standardized starting position that makes comparison between experiments more reliable.

Hence a multi-compartment microfluidic device for enabling fluidic isolation among interconnected compartments and positioning biological specimens within the compartments of the device is described. The claims, however and the full scope of their equivalents are what define the boundaries of the invention.

What is claimed is:

1. A multi-compartment microfluidic device for enabling fluidic isolation among interconnected compartments and accomplishing centrifugal positioning of biological specimens within the device comprising:
a micropatterned substrate coupled with an optically transparent housing;
said optically transparent housing comprising a first microfluidic region having a first entry reservoir for accepting a first volume of fluid;
said optically transparent housing further comprising a second microfluidic region having a second entry reservoir for accepting a second volume of fluid that is less than said first volume of fluid to create hydrostatic pressure;
a barrier region that couples said first microfluidic region with said second microfluidic region to enable a biological specimen to extend across said first microfluidic region, said barrier region and said second microfluidic region; and
said barrier region comprising at least one embedded microgroove having a width and height that enables said second volume of fluid to be fluidically isolated from said first volume of fluid via said hydrostatic pressure maintained via said at least one embedded microgroove where cells are aligned to a chosen side of said first microfluidic region through the use of centrifugal force.

2. A multi-compartment microfluidic device for enabling fluidic isolation among interconnected compartments and accomplishing positioning of biological specimens within the device via substrate patterning comprising:
a micropatterned substrate coupled with an optically transparent housing;
said optically transparent housing comprising a first microfluidic region having a first entry reservoir for accepting a first volume of fluid;
said optically transparent housing further comprising a second microfluidic region having a second entry reservoir for accepting a second volume of fluid that is less than said first volume of fluid to create hydrostatic pressure;
a barrier region that couples said first microfluidic region with said second microfluidic region to enable a biological specimen to extend across said first microfluidic region, said barrier region and said second microfluidic region; and
said barrier region comprising at least one embedded microgroove having a width and height that enables said second volume of fluid to be fluidically isolated from said first volume of fluid via said hydrostatic pressure maintained via said at least one embedded microgroove where cells are aligned to a specific location through the use of substrate patterning.
